(19) United States
(12) Patent Application Publication
(10) Pub. No.: US 2007/0148326 A1
(43) Pub. Date: Jun. 28, 2007

(54) Syringe

(76) Inventors: Mitchell R. Hastings, Rancho Murieta, CA (US); Thomas Michael Logan, Newtown Square, PA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/320,941

(22) Filed: Dec. 28, 2005

(51) Int. Cl. A61L 27/32; A61M 5/315; A61M 25/00; A61M 5/32; B05D 3/00

(52) U.S. Cl. 427/2.27; 604/265; 604/230

(57) ABSTRACT

The present invention relates to a syringe including a barrel and a plunger. The barrel, the plunger, or both the barrel and the plunger can be made from or coated with a material that provides advantageous function and/or durability of these components. In certain embodiments, the barrel, the plunger, or both the barrel and the plunger can include or can be coated with a silicon material, such as silicon carbide or silicon oxide, or with a siloxane, such as a polymeric siloxane. In certain embodiments, the plunger can include or can be coated with a steel more durable than stainless steel. The invention also includes systems and methods employing such a syringe.

### BACKGROUND OF THE INVENTION

Many conventional syringes are made with borosilicate glass barrels and stainless steel plungers. Such a syringe can provide adequate function for samples in organic solvents such as hexane, toluene, or methylene chloride. However, other solvents, such as higher viscosity solvents, can compromise syringe function, for example, by causing binding, bending, or oxidation of the plunger. Loss of function of a syringe in a device such as an autosampler can ruin or waste samples that may have been unique or costly to prepare. Accordingly, there remains a need for more suitable syringes.

### SUMMARY

The present invention relates to a syringe including a barrel and a plunger. The barrel, the plunger, or both the barrel and the plunger can be made from or coated with a material that provides advantageous function and/or durability of these components. In certain embodiments, the barrel, the plunger, or both the barrel and the plunger can include or can be coated with a silicon material, such as silicon carbide or silicon oxide, or with a siloxane, such as a polymeric siloxane. In certain embodiments, the plunger can include or can be coated with a steel more durable than stainless steel. The invention also includes systems and methods employing such a syringe.

In an embodiment, the present invention includes a syringe including a barrel and a plunger. The barrel defines a cavity, which is configured for containing a fluid. At least a portion of the surface of the barrel defining the cavity can include a coating of a silicon or siloxane material. The plunger and barrel are configured for drawing fluid into the cavity and expelling liquid from the cavity.

In an embodiment, the present invention includes a syringe including a barrel and a plunger. The barrel defines a cavity, which is configured for containing a fluid. The plunger includes a fluid contact surface. The plunger can include superalloy. At least a portion of the fluid contact surface of the plunger can include a coating of a silicon or siloxane material. Or, the plunger can include the coating and the superalloy. The plunger and barrel are configured for drawing fluid into the cavity and expelling liquid from the cavity.

In an embodiment, the present invention includes a system or apparatus including the present syringe. In an embodiment, the system can be an autosampler. The autosampler can be configured to operate the syringe and to provide a sample to a chromatography apparatus.

In an embodiment, the present invention includes a method employing the present syringe. The method can include drawing a sample including a protic solvent into the present syringe. The method can also include introducing the sample into a chromatography system.

### BRIEF DESCRIPTION OF THE FIGURES

FIG. 1 schematically illustrates a cross sectional view of an embodiment of a syringe according to the present invention.

FIG. 2 schematically illustrates a cross sectional view of an embodiment of a syringe according to the present invention.

FIG. 3 schematically illustrates a cross sectional view of an embodiment of a syringe according to the present invention.

FIG. 4 schematically illustrates a cross sectional view of an embodiment of a syringe according to the present invention.

FIG. 5 schematically illustrates a cross sectional view of an embodiment of a syringe according to the present invention.

FIG. 6 schematically illustrates an embodiment of a system including a syringe according to the present invention.

FIG. 7 schematically illustrates a cross sectional view of an embodiment of a syringe according to the present invention and a vial.

### DETAILED DESCRIPTION OF EMBODIMENTS OF THE INVENTION

### Definitions

Unless defined otherwise below, all technical and scientific terms used herein have the same meaning as commonly understood by one of ordinary skill in the art to which this invention belongs. Still, certain terms are defined herein for the sake of clarity.

As used herein, the term “syringe” refers to a fluid handling system including a barrel and a plunger. A syringe can be employed to take up fluids into the syringe or withdraw fluids from an object. A syringe can be employed to expel fluid from the syringe or to inject fluid into an object. In an embodiment, a syringe can include a hollow barrel fitted with a plunger and a hollow probe or needle. In an embodiment, a syringe can include a plunger fitted to a tube (e.g., the barrel). The tube or barrel can have a small opening on one end, which can provide fluid communication between a cavity defined by the tube or barrel and the surroundings. In an embodiment, a syringe can operate on the principle of suction by filling the barrel with a fluid at the opening when the plunger is drawn out, and expelling the substance when the plunger is depressed.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

It will also be appreciated that throughout the present application that words such as “upper” and “lower” are used in a relative sense only.

Various embodiments of the present invention will be described in detail with reference to the drawings, wherein like reference numerals represent like parts throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

Syringe

An embodiment of the present invention relates to a syringe including a barrel and a plunger. The barrel, the plunger, or both the barrel and the plunger can be made from or coated with a material that provides advantageous function and/or durability of these components. For example, the barrel and/or plunger can include a coating or material that can provide advantageous function for samples in solvents that are polar or protic or that have increased viscosity or increased surface tension. For example, the barrel and/or plunger can include a coating or material that can provide advantageous function for aqueous samples.

The barrel can be made of any of a variety of materials, such as glass, for example borosilicate glass. The barrel can include or can be coated with any of a variety of materials, such as silicon or siloxane materials. For example, a glass barrel can be coated with a silicon material, such as silicon carbide or silicon oxide, or with a siloxane, such as a polymeric siloxane. Suitable polymeric siloxanes include polymethylhydrosiloxane. Any of a variety of known methods can be employed for applying the silicon or siloxane material to the barrel.

The barrel of a syringe forms a cavity that can contain a fluid. An interior surface of the barrel lines the cavity. All or a portion of that interior surface can be coated with a material, such as a silicon or siloxane material. In an embodiment, the portion of the interior surface of the barrel that contacts or that can contact a fluid during operation of the syringe can be coated with the material, such as a silicon or siloxane material.

The plunger can be made of any of a variety of materials, such as steel, for example, stainless steel. In an embodiment, the plunger can include or can be coated with a steel more durable than stainless steel (e.g., a superalloy, such as a nickel-based superalloy). In an embodiment, the plunger can include or can be coated with any of a variety of materials, such as silicon or siloxane materials. For example, a steel plunger can be coated with a silicon material, such as silicon carbide or silicon oxide, or with a siloxane, such as a polymeric siloxane. Suitable polymeric siloxanes include polymethylhydrosiloxane. Any of a variety of known methods can be employed for applying the silicon or siloxane material to the plunger.

The plunger functions in a cavity in the barrel of the syringe that can contain a fluid. At least a portion of the exterior surface of the plunger is in fluid communication with the cavity. All or a portion of that exterior surface can be coated with or made of a superalloy. In an embodiment, the portion of the exterior surface of the plunger that contacts or that can contact a fluid during operation of the syringe can be coated with or made of a superalloy. All or a portion of that exterior surface can be coated with a material, such as a silicon or siloxane material. In an embodiment, the portion of the exterior surface of the plunger that contacts or that can contact a fluid during operation of the syringe can be coated with the material, such as a silicon or siloxane material.

The present syringe can be manually operated or can be a component of an apparatus such as an autosampler for chromatography. An autosampler including the present syringe can be employed for providing samples to chromatography apparatus such as gas chromatography apparatus, gas-liquid chromatography apparatus, high-pressure liquid chromatography apparatus, and the like. The present syringe as a component of an autosampler includes a needle suitable for entering a vial, for example, by piercing a septum. As a component of an autosampler, the present syringe can also include a plunger with a portion configured for coupling to or interacting with the autosampler. The autosampler operates the plunger to pull a sample into and expel a sample from the present syringe. The autosampler can move the syringe relative to a vial to introduce the needle into a sample in the vial.

The present syringe can be employed in a method for introducing a sample into chromatography apparatus such as gas chromatography apparatus, gas-liquid chromatography apparatus, high-pressure liquid chromatography apparatus, and the like. The method can include introducing a sample into the syringe and expelling the sample from the syringe. The sample can contact the barrel and the plunger of the syringe. The sample can include a polar solvent or a protic solvent. The polar solvent can be more polar than hexane, toluene, and methylene chloride. The sample can have viscosity greater than hexane, toluene, and methylene chloride. The sample can have surface tension greater than hexane, toluene, and methylene chloride.

In an embodiment, the method can include introducing and expelling such a sample more times than could be accomplished with a conventional syringe. In an embodiment, the method can include introducing and expelling such a sample at least about twice as many times as the number of injections from an otherwise equivalent conventional syringe under conditions where water content in the solvent is high or exclusively water.

### Embodiments of the Syringe

In an embodiment, the syringe can include a glass barrel coated with silicon carbide and a plunger including steel more durable than stainless steel (e.g., a superalloy, such as a nickel-based superalloy). In an embodiment, the syringe can include a glass barrel coated with silicon carbide and a plunger coated with silicon carbide. In an embodiment, the syringe can include a glass barrel coated with silicon carbide and a plunger coated with silicon oxide. In an embodiment, the syringe can include a glass barrel coated with silicon carbide and a plunger coated with polymethylhydrosiloxane.

In an embodiment, the syringe can include a glass barrel coated with silicon oxide and a plunger including steel more durable than stainless steel (e.g., a superalloy, such as a nickel-based superalloy). In an embodiment, the syringe can include a glass barrel coated with silicon oxide and a plunger coated with silicon carbide. In an embodiment, the syringe can include a glass barrel coated with silicon oxide and a plunger coated with silicon oxide. In an embodiment, the syringe can include a glass barrel coated with silicon oxide and a plunger coated with polymethylhydrosiloxane.

In an embodiment, the syringe can include a glass barrel coated with polymethylhydrosiloxane and a plunger including steel more durable than stainless steel (e.g., a superalloy, such as a nickel-based superalloy). In an embodiment, the syringe can include a glass barrel coated with polymethylhydrosiloxane and a plunger coated with silicon carbide. In an embodiment, the syringe can include a glass barrel coated with polymethylhydrosiloxane and a plunger coated with silicon oxide. In an embodiment, the syringe can include a glass barrel coated with polymethylhydrosiloxane and a plunger coated with polymethylhydrosiloxane.

### Illustrated Embodiments

Embodiments of the present syringe, present apparatus including the syringe, and present method can be described with reference to the Figures.

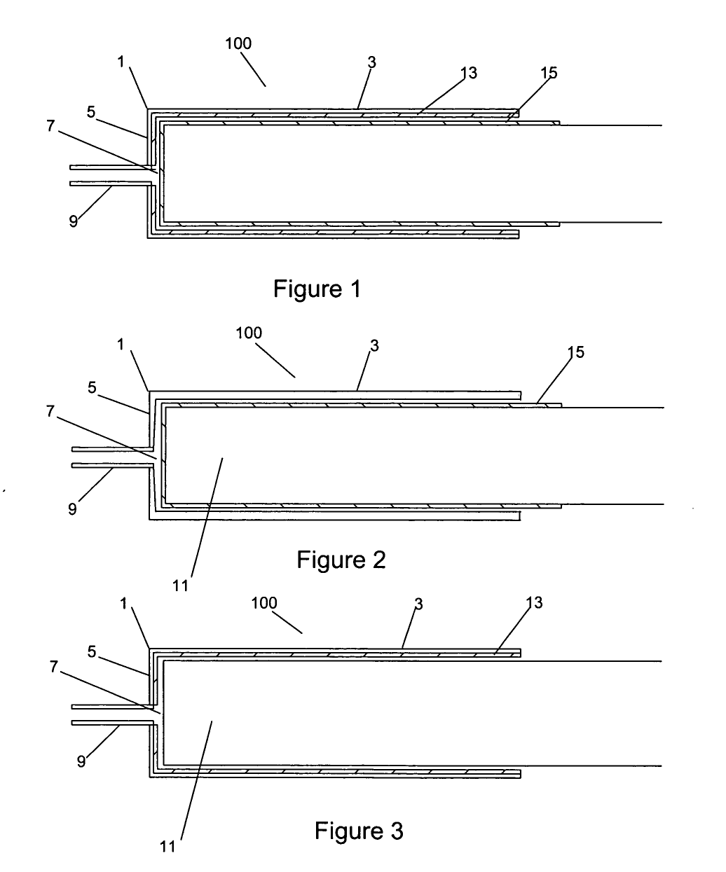

The illustrated embodiment of the syringe, syringe 100, can include body 1 as an embodiment of the barrel (FIGS. 1-5). Body 1 can include cylindrical side wall 3 and base 5. Body 1 defines chamber 7 (FIGS. 2-4). Probe 9, an embodiment of the needle, is coupled to base 5 of body 1 and can provide fluid communication from the surroundings into chamber 7. Body 1 can be made of any of a variety of materials, such as glass, for example borosilicate glass.

The present syringe can include piston 11 as an embodiment of the plunger (FIGS. 1-4). Piston 11 can be configured to be housed in body 1 and body 1 can be configured to house piston 11. The shapes of piston 11 and body 1 can conform within tolerances effective for drawing fluid into chamber 7 through probe 9 upon withdrawing piston 11 from body 1 and for expelling fluid from chamber 7 through probe 9 upon moving piston 11 toward base 5 of body 1. In an embodiment, the tolerances can be measured in microns.

Referring now to FIG. 1, body 1 can include a first layer 13 as an embodiment of the coating. First layer 13 can be or include a material that provides advantageous function and/or durability to body 1, for example, with aqueous samples. First layer 13 can be or include a silicon material, such as silicon carbide or silicon oxide, or with a siloxane, such as a polymeric siloxane. Suitable polymeric siloxanes include polymethylhydrosiloxane.

Still referring to FIG. 1, piston 11 can include a second layer 15 as an embodiment of the coating. Second layer 15 can be or include a material that provides advantageous function and/or durability to the piston, for example, with aqueous samples. Second layer 15 can be or include a silicon material, such as silicon carbide or silicon oxide, or with a siloxane, such as a polymeric siloxane. Suitable polymeric siloxanes include polymethylhydrosiloxane. Piston 11 can be made of any of a variety of materials, such as steel, for example, stainless steel. In an embodiment, piston 11 can be made of, can include, or can be coated with a steel more durable than stainless steel (e.g., a superalloy, such as a nickel-based superalloy).

As illustrated in FIG. 2, in an embodiment, body 1 can lack first layer 13. In the illustrated embodiment, piston 11 includes second layer 15. Second layer 15 can be as described above with reference to FIG. 1. Piston 11 can be made of any of a variety of materials, as described above with reference to FIG. 1.

As illustrated in FIG. 3, in an embodiment, piston 11 can lack second layer 15. In the illustrated embodiment, body 1 includes first layer 13. First layer 13 can be as described above with reference to FIG. 1. Piston 11 can be made of any of a variety of materials, as described above with reference to FIG. 1. In an embodiment, piston 11 can be made of, can include, or can be coated with a steel more durable than stainless steel (e.g., a superalloy, such as a nickel-based superalloy).

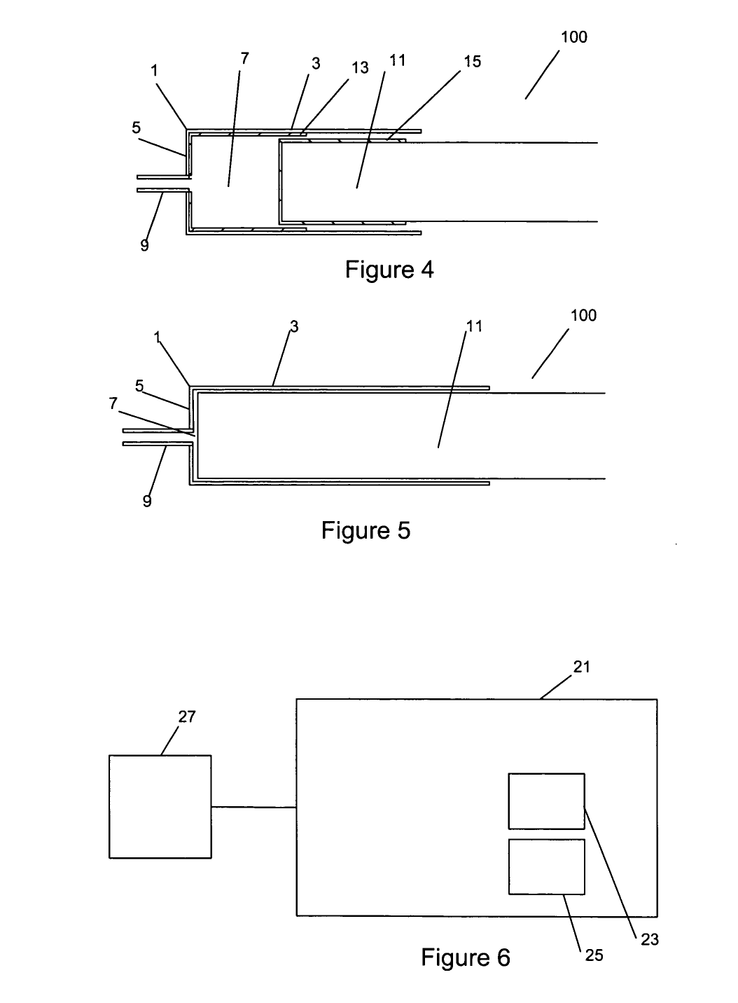

FIG. 4 illustrates an embodiment of body 1 including first layer 13 extending over only a portion of the interior surface of body 1 that defines chamber 7. This Figure also illustrates an embodiment of piston 11 including second layer 15 extending over only a portion of the exterior surface of piston 11. In an embodiment, the portion of piston 11 indicated in FIG. 4 as coextensive with second layer 15 can be can be made of, can include, or can be coated with a steel more durable than stainless steel (e.g., a superalloy, such as a nickel-based superalloy).

As illustrated in FIG. 5, in an embodiment, body 1 can lack first layer 13 and piston 11 can lack second layer 15. In this illustrated embodiment, piston 11 is made of, includes, or is coated with a steel more durable than stainless steel (e.g., a superalloy, such as a nickel-based superalloy).

As schematically illustrated in FIG. 6, the present syringe can be a component of an apparatus. In the illustrated embodiment, the apparatus is an autosampler 21 for chromatography. FIG. 6 schematically illustrates autosampler 21 including sampler syringe 23 as an embodiment of the present syringe and a sample vial 25. Autosampler 21 is shown as functionally coupled to chromatography system 27. Sampler syringe 23 can include any of the features illustrated in FIGS. 1-5. Sampler syringe 23 is configured to be operated by autosampler 21 for taking up a sample from sample vial 25 and discharging the sample into chromatography system 27. Sampler syringe 23 and autosampler 21 can be configured to move sampler syringe 23 relative to the sample vial 25 to introduce the probe 9 into a sample in the vial.

Chromatography system 27 can be any of a variety of chromatography systems that employ an autosampler, for example, gas chromatography system, gas-liquid chromatography system, high-pressure liquid chromatography system, and the like.

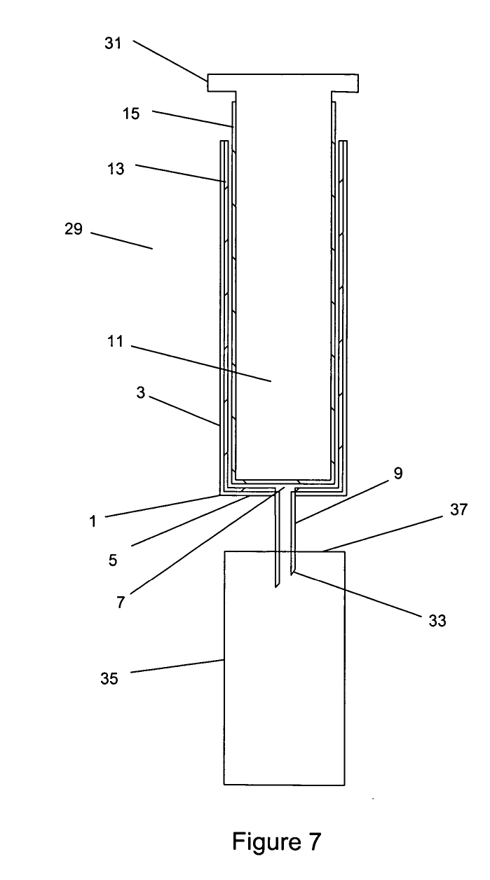

FIG. 7 schematically illustrates system syringe 29 as an embodiment of sampler syringe 23. System syringe 29 includes body 1, cylindrical side wall 3, base 5, chamber 7, probe 9, and piston 11 as described above with reference to FIGS. 1-5. Similarly, system syringe 29 can include first layer 13 and/or second layer 15 in configurations as described above with reference to FIGS. 1-5.

As illustrated, system syringe 29 also includes piston flange 31. Piston flange 31 represents an embodiment of a portion of piston 11 configured for coupling to or interacting with an autosampler. For example, piston flange 31 can mate with a slot on a movable member of an autosampler for moving piston 11 relative to body I of system syringe 29. For example, the autosampler member can move away from syringe body 1 along the major axis of piston 11 and body 1 to pull a sample into system syringe 29. For example, the autosampler member can move toward from syringe body 1 along the major axis of piston 11 and body 1 to expel a sample from system syringe 29. Any of the embodiments of the present syringe illustrated in FIGS. 1-5 can also include piston flange 31.

As illustrated, system syringe 29 also includes beveled probe 33, an embodiment of probe 9. Beveled probe 33 as illustrated schematically represents a pointed or sharpened needle, such as those commonly employed on a syringe configured to puncture a septum or seal on a bottle or vial. As schematically illustrated in FIG. 7, beveled probe 33 is partly within vial 35 and has pierced septum 37. Any of the embodiments of the present syringe illustrated in FIGS. 1-5 can include beveled probe 33.

Steel

The plunger can include or can be coated with a steel more durable than stainless steel (e.g., a superalloy, such as a nickel-based superalloy). Suitable steels include an austenitic nickel-chromium-iron alloy which can contain a higher level of nickel and/or chromium than stainless steel. The steel can also include small quantities of other elements, such as molybdenum. Such steel is those sold under the tradename INCONEL® steel.

As used herein, the term superalloy, refers to an alloy with, compared to conventional stainless steel, enhanced mechanical strength, good surface stability, corrosion resistance, and that can withstand high temperatures without oxidizing or losing mechanical properties. Superalloys can be based on nickel, cobalt, or iron and can also include chromium, molybdenum, tungsten, aluminum, zirconium, niobium, rhenium, carbon or silicon are just a few examples. Superalloys include those alloys sold under the tradenames Hastelloy, Inconel, MP98T, TMS-63, TMS-71, and TMS-75.

In a superalloy, molybdenum (up to 5%) can strengthen the nickel matrix and extend service temperatures, for example, by partitioning between the nickel matrix and the gamma prime precipitate phase. Higher Mo alloys, e.g., 9% Mo, can also be used. Molybdenum enhances the corrosion resistance and mechanical properties of nickel base alloys in the same way that it improves the corrosion resistance of stainless steels. Suitable alloys include a 5% molybdenum cobalt base investment casting alloy, such as that sold under the tradename Stellite 21. Such an alloy can exhibit suitable corrosion resistance to body fluids.

Suitable superalloys include those listed in Tables 1 and 2. The superalloys listed in Table 1 are available under the tradename INCONEL® steel.

TABLE 1
Suitable Superalloys

| Type | % Ni | % Cr | % C | % Mn | % Si | % Fe | % S | % Cu | % Al | % Ti | T P | % Co | % Nb | % B | % Mo |
|---|---|---|---|---|---|---|---|---|---|---|---|---|---|---|---|
| 600 | 72 | 14-17 | 0.15 | 1 | 0.5 | 6-10 | 0.015 | 0.5 | 0 | 0 | 0 | 0 | 0 | 0 | 0 |
| | min | | max | max | max | | max | max | | | | | | | |
| 601 | 58-63 | 21-25 | 0.1 | 1 | 0.5 | bal | 0.015 | 1 | 1-1.7 | 0 | 0 | 0 | 0 | 0 | 0 |
| | | | max | max | max | | max | max | | | | | | | |
| 625 | 58 | 20-23 | 0.1 | 0.5 | 0.5 | 5 | 0.015 | 0 | 0.4 | 0.4 | 0.015 | 1 | 3.15-4.15 | 0 | 8-10 |
| | min | | max | max | max | max | max | | max | max | max | max | | | |
| 718 | 50-55 | 17-21 | 0.08 | 0.35 | 0.35 | bal | 0.015 | 0.3 | 0.2-0.8 | 0.65-1.15 | 0.015 | 1 | 4.75-5.5 | 0.006 | 2.8-3.3 |
| | | | max | max | max | | max | max | | | | max | | max | |
| 800 | 32.5 | 21 | 0.1 | 0.8 | 0.008 | 46 | 0 | 0.4 | 0.4 | 0.4 | 0 | 0 | 0 | 0 | 0 |
| | | | max | max | max | | | | | | | | | | |

TABLE 2
Additional Suitable Superalloys

| | | | | | | | | | | | Melting |
|---|---|---|---|---|---|---|---|---|---|---|---|
| Alloy | C | Mn | Si | Cr | Ni | Mo | W | Co | Fe | Other | Range ° F. |
| Ni Alloy B | .12 | 1 | 1 | 1 | Bal | 26-30 | | 2.5 | 4-7 | P, .03 Max.; | 2375-2495 |
| ASTM 5396B | | | | | | | | | | Sc .03 Max.; | |
| | | | | | | | | | | V, .2-.6 | |
| ASTM A-494 | .12 | 1 | 1 | 1 | Bal | 26-30 | | | 4-6 | P, .04 Max.; | 2375-2495 |
| GR N-12MV | | | | | | | | | | Sc .03 Max.; | |
| | | | | | | | | | | V, .2-.6 | |
| ASTM A-494 | .07 | 1 | 1 | 1 | Bal | 30-33 | | | 3 | P, .04 Max.; | 2375-2495 |
| GR N-7M | | | | | | | | | | Sc .03 Max. | |
| Ni Alloy C | .15 | 1 | 1 | 15.5-17.5 | Bal | 16-18 | 3.75-5.25 | 2.5 | 4.5-7 | V, .2:6; | 2310-2450 |
| ASTM 5388E | | | | | | | | | | P, .03 Max.; | |
| | | | | | | | | | | S, .03 Max. | |
| AMS 5389B | .15 | 1 | 1 | 15.5-17.5 | Bal | 16-18 | 3.75-5.25 | 2.5 | 4.5-7 | V, .2-.6; | 2310-2450 |
| | | | | | | | | | | P, .04 Max.; | |
| | | | | | | | | | | S, .04 Max. | |
| ASTM A-494 | .12 | 1 | 1 | 15.5-.5 | Bal | 16-18 | 3.75-5.25 | | 4.5-7.5 | P, .04 Max.; | 2310-2450 |
| GR CW-12 MW | | | | | | | | | | S, .03 Max.; | |
| | | | | | | | | | | V, .2-.4 Max. | |
| ASTM A-494 | .07 | 1 | 1 | 17-20 | Bal | 17-20 | | | 3 | P, .04 Max.; | 2310-2450 |
| CW-6M | | | | | | | | | | S, .03 Max. | |
| ASTM A-494 | .02 | 1 | .8 | 15-17.5 | Bal | 15-17.5 | 1 | | 2 | P, .03 Max.; | 2310-2450 |
| GR CW-2M | | | | | | | | | | S, .03 Max. | |
| Ni Alloy D | .12 | .5-1.25 | 8.5-10 | 1 | Bal | | | 1.5 | 2 | Cu, 2-4 | 2030-2050 |
| Ni Alloy F | .12 | 1-2 | 1 | 21-23 | 44-47 | 5.5-7.5 | 1 | 2.5 | Bal | P, .04 Max.; | 2325-2375 |
| | | | | | | | | | | S, .03 Max.; | |
| | | | | | | | | | | Cb/Ta, 1.75-2.5 | |
| Ni Alloy G | .12 | 1-2 | 1 | 21-23.5 | Bal | 5.5-7.5 | 1 | 2.5 | 18-21 | P, .04 Max.; | 2300-2450 |
| | | | | | | | | | | S, .03 Max.; | |
| | | | | | | | | | | Cu, 1.5-2.5; | |
| | | | | | | | | | | Cb/Ta, 1.75-2.5 | |
| Ni Alloy N | .04-.1 | .8 | 1 | 6-8 | Bal | 15-18 | .5 | .2 | 5 | P, .015 Max.; | 2375-2450 |
| | | | | | | | | | | S, .02 Max.; | |
| | | | | | | | | | | Cu, .35 Max. | |
| | | | | | | | | | | B, .01 Max.; | |
| | | | | | | | | | | Al + Ti, .5 Max. | |
| Ni Alloy X | .1 | 1 | 1 | 20.5-23 | Bal | 8-10 | .2-1 | .5-2.5 | 17-20 | P, .04 Max.; | 2300-2470 |
| ASTM 5390C | | | | | | | | | | S, .03 Max.; | |
| | | | | | | | | | | B, .01 Max. | |
| | | | | | | | | | | Se,. 005 Max. | |
| Ni Alloy 210 | 1 | 1.5 | 2 | | Bal 95 | | | | 3 | P, .03 Max.; | 2450-2600 |
| ASTM A- | | | | | | | | | | S, .03 Max.; | |
| 494 GR CZ100 | | | | | | | | | | Cu, 1.25 Max. | |
| Ni Alloy 213 | 1.-2.5 | 1.5 | 2 | | Bal | | | | 1.25 | S, .015 Max.; | 2400-2600 |
| | | | | | | | | | | Cu, 1.25 Max. | |
| Ni Alloy 305 | 1 | 1.5 | 5.5-6.5 | | Bal | | | | 1.25 | S, .015 Max.; | 2400-2600 |
| | | | | | | | | | | Cu, 1.25 Max. | |
| Ni—Cr Alloy | .4 | 1.5 | 3 | 14-17 | Bal | | | | 11 | P, .03 Max.; | 2540-2610 |
| 610, ASTM A- | | | | | | | | | | S, .03 Max. | |
| 494, GR CY40 | | | | | | | | | | | |
| Ni—Cr Alloy | .1 | .3 | 1 | 48-52 | Bal | | | | 1 | P, .02 Max.; | 2440-2470 |
| ASTM A-560 | | | | | | | | | | S, .02 Max.; | |
| 50Cr—50Ni | | | | | | | | | | Al, .25 Max.; | |
| | | | | | | | | | | Ti, .5 Max.; | |
| | | | | | | | | | | N₂, .3 Max. | |
| Ni—Cr Alloy | .1 | .3 | 1 | 58-62 | Bal | | | | 1 | P, .02 Max.; | 2580-2610 |
| ASTM A-560 | | | | | | | | | | S, .02 Max.; | |
| 60CR—40Ni | | | | | | | | | | Al, .25 Max.; | |
| | | | | | | | | | | Ti, .5 Max.; | |
| | | | | | | | | | | N₂, .3 Max. | |
| Ni—Cr Alloy | .15 | 1 | .5 | 14-17 | 72 Min. | | | | 6-10 | Cu, .5 Max.; | 2540-2610 |
| 600IC | | | | | | | | | | P, .03 Max.; | |
| ALLOY 600 | | | | | | | | | | S, .015 Max. | |
| Ni—Cr | .4 | 1.5 | 3 | 14-17 | Bal | | | | 11 | Cu, 1.25 Max. | 2540-2600 |
| Alloy 610 | | | | | | | | | | | |
| CY 40 | .4 | 1.5 | 3 | 14-17 | Bal | | | | 11 | Cu, .5 Max. | 2540-2610 |
| Ni—Cr | .4 | 1.5 | 2 | 14-17 | Bal | | | | 11 | Cu, .5 Max.; | 2540-2600 |
| Alloy 611 | | | | | | | | | | Cb/Ta, 1-3 | |
| Ni—Cr—Mo | .1 | .5 | .5 | 20-23 | Bal | 8-10 | | 1 | 5 | Cb, 3.15-4.15; | 2325-2375 |
| AMS 5402B | | | | | | | | | | Cu, .3 Max.; | |
| | | | | | | | | | | Al, .1 Max.; | |
| | | | | | | | | | | Ti, .1 Max.; | |
| | | | | | | | | | | P, .03 Max.; | |
| | | | | | | | | | | S, .04 Max.; | |
| | | | | | | | | | | Ta, .05 Max. | |
| Alloy 625 | .06 | 1 | 1 | 20-23 | Bal | 8-10 | | | 5 | P, .015 Max.; | 2325-2375 |
| ASTMA-494 | | | | | | | | | | S, .015 Max.; | |
| GR CW-6MC | | | | | | | | | | Cb, 3.15-4.5 | |
| Ni—Cr Alloy | .1 | .3 | .5 | 48-52 | Bal | | | Cb | 1 | S, .02 Max.; | 2370-2410 |
| 657 | | | | | | | | 1.4-1.7 | | P, .02 Max.; | |
| ASTM A-560 | | | | | | | | | | Al, .25 Max.; | |
| GR | | | | | | | | | | Ti, .5 Max.; | |
| 50Cr—50Ni— | | | | | | | | | | N₂, .16 Max.; | |
| Cb | | | | | | | | | | C + N₂, .2 Max. | |
| Ni—Cr Alloy | .1-.4 | 1.5 | 5-6 | 14-17 | Bal | | | | 11 | Cu, .5 Max. | 2540-2600 |
| 705 S | .1-.4 | 1.5 | 5-6 | 14-17 | Bal | | | | 11 | S, .015 Max.; | 2540-2600 |
| | | | | | | | | | | Cu, 1.25 Max. | |
| Ni—Cr—Mo | .04-.08 | | 3-3.5 | 14.5-15.5 | 48-51 | 31-33 | | | | P, .03 Max.; | 2250-2900 |
| Alloy 700 | | | | | | | | | | S, .03 Max.; | |
| | | | | | | | | | | Fe + Co, 3 Max.; | |
| | | | | | | | | | | N₂, .05 Max.; | |
| | | | | | | | | | | O₂, .05 Max. | |
| N—Cr | .05 | 1.5 | 0.5 | 11-14 | Bal | 2-3.5 | | | 2 | P, .03 Max.; | 2250-2400 |
| Alloy 88 | | | | | | | | | | | |
| ASTM A-494 | | | | 14 | | | | | | S, .03 Max. | |
| GR CY5SnBiM | | | | | | | | | | Sn, 3-5 | |
| | | | | | | | | | | Bi, 3-5 | |
| Ni—Co Alloy | .03 | .1 | .1 | | 16-17.5 | 4.4-4.8 | | 9.5-11 | | P, .01 Max.; | 2350-2500 |
| Maraging | | | | | | | | | | S, .01 Max.; | |
| AMS 5339B | | | | | | | | | | Ti, .15-.45; | |
| | | | | | | | | | | Al, .02-.10 | |
| Ni—Cu Alloy | .35 | 1.5 | 1.25 | | Bal | | | | 3.5 | Cu, 26-33; | 2400-2450 |
| M-35 ASTM | | | | | | | | | | P, .03 Max.; | |
| A-494 GR | | | | | | | | | | S, .03 Max. | |
| M-35p-1(a) | | | | | | | | | | | |
| ASTM A-494 | .35 | 1.5 | 2 | | Bal | | | | 3.5 | Cu, 26-33; | 2400-2450 |
| GR; M-35-2 | | | | | | | | | | P, .03 Max.; | |
| | | | | | | | | | | S, .03 Max. | |
| Ni—Cu | .3 | 1.5 | 2.5-3.5 | | 62-68 | | | | 3 | Cu, Bal | 2370-2460 |
| Alloy GR I | | | | | | | | | | | |
| MIL-N-4498 | .25 | 1.5 | 3.5-5 | | 62-68 | | | | 3.5 | Cu, Bal | 2370-2460 |
| GR II | | | | | | | | | | | |
| Comp. B | .3 | 1.5 | 2.7-3.7 | | 61-68 | | | | 2.5 | Cu, 27-33 | 2350-2400 |
| (Alloy 506) | | | | | | | | | | Al, .5 Max. | |
| Comp. C | .2 | 1.5 | 3.3-4.3 | | 60 | | | | 2.5 | Cu, 27-31 | 2300-2350 |
| (Alloy 505) | | | | | | | | | | Al, .5 Max. | |
| Comp. D | .25 | 1.5 | 3.5-4.5 | | 60 | | | | 2.5 | Cu, 27-31 | 2300-2350 |
| (Alloy s) | | | | | | | | | | Al, .5 Max. | |
| Comp. E | .3 | 1.5 | 1-2 | | Bal | | | | 3.5 | Cu, 26-33; | 2300-2350 |
| (Alloy 411) | | | | | | | | | | Al, .5 Max. | |
| | | | | | | | | | | Cb/Ta, 1-3 | |
| Comp. F | .4-.7 | 1.50 | 2.3-3 | | Bal | | | 1 | 2.5 | Cu, 29-34; | 2375-2425 |
| (Alloy RH) | | | | | | | | | | Al, .5 Max. | |
| Ni—Cu | .3 | 1.5 | 1-2 | | Bal | | | | 3.5 | Cu, 26-33; | 2370-2460 |
| Alloy ASTM | | | | | | | | | | Cb, 1-3; | |
| A-494 M-30 C | | | | | | | | | | P, .03 Max.; | |
| | | | | | | | | | | S, .03 Max. | |
| ASTM A-494 | .3 | 1.5 | 2.7-3.7 | | Bal | | | | 3.5 | Cu, 27-33; | 2350-2400 |
| M-30 H | | | | | | | | | | P, .03 Max. | |
| | | | | | | | | | | S, .03 Max. | |
| ASTM A-494 | .25 | 1.5 | 3.5-4.5 | | Bal | | | | 3.5 | Cu, 27-33; | 2300-2350 |
| M-25 S | | | | | | | | | | P, .03 Max. | |
| | | | | | | | | | | S, .03 Max. | |
| Comp. M-30 C | .3 | 1.5 | 1-2 | | Bal | | | | 2.5 | Cu, 26-33; | 2370-2460 |
| MIL-C-4723 | | | | | | | | | | Cb, 1-3; | |
| | | | | | | | | | | P, .03 Max.; | |
| | | | | | | | | | | S, .03 Max. | |
| Comp. M-30 H | .3 | 1.5 | 2.7-3.7 | | Bal | | | | 2.5 | Cu, 27-33; | 2350-2400 |
| MIL-C-24723 | | | | | | | | | | P, .03 Max.; | |
| | | | | | | | | | | S, .03 Max. | |
| Comp. M-25 S | .25 | 1.5 | 3.5-4.5 | | Bal | | | | 2.5 | Cu, 27-33; | 2300-2350 |
| MIL-C-24723 | | | | | | | | | | P, .03 Max.; | |
| | | | | | | | | | | S, .03 Max. | |

Silicon and Siloxane Materials

The present syringe can include or can be coated with any of a variety of silicon or siloxane materials. For example, the barrel and/or plunger can be coated with a silicon material, such as silicon carbide or silicon oxide, or with a siloxane, such as a polymeric siloxane. Suitable polymeric siloxanes include polymethylhydrosiloxane. Any of a variety of known methods can be employed to apply the silicon or siloxane material to the barrel and/or plunger.

The following general formula represents a family of suitable methylhydrosilicone homopolymers: 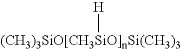
Such a homopolymer, e.g., polymethylhydrosiloxane, can be coupled to the barrel and/or plunger. Polymethylhydrosiloxane has CAS no. 9004-73-73.

Although not limiting to the present invention, it is believed that such a polymer can react with hydroxyl active materials in the presence of tin octoate, zinc octoate and a variety of other metal salt catalysts to form bonds with the evolution of hydrogen. This reaction can be used to couple the polymer to materials such as glass. The reaction can be accomplished in dilute (0.5-2.0%) solutions in hydrocarbons or chlorinated solvents. Such a coating can be cured at 110° -150°. Suitable accelerators include dibutyltindilaurate and zinc, iron or tin octoates.

As used herein, the term “silicon carbide” refers-to a compound of silicon and carbon represented by the chemical formula SiC, which can be a ceramic. Silicon carbide can take the form of an extremely hard, dark, iridescent crystal that is insoluble in water and other common solvents. Very pure silicon carbide is white or colorless. Silicon carbide is also known as carborundum or moissanite.

Silicon carbide can be prepared and applied to surfaces such as the present barrel or plunger by chemical vapor deposition. Commercial production can be by fusing sand and carbon at a high temperature, between 1600° C. and 2500° C. Sintered SiC can be produced from pure SiC powder with non-oxide sintering aids. Conventional ceramic forming processes can be used and the material is sintered in an inert atmosphere at temperatures up to 2000° C. or higher. Reaction bonded SiC can be made by infiltrating compacts made of mixtures of SiC and carbon with liquid silicon.

Silicon carbide exhibits one-dimensional polymorphism (e.g., polytypsim) and crystallizes in many polytypes with different stacking sequence of the double layer. Silicon carbide exhibits advantageous mechanical hardness and chemical inertness.

As used herein, the terms “silicon dioxide” and “silicon oxide” refer to the compound of chemical formula SiO₂. Suitable silicon oxide includes fused silica. Suitable silicon oxide includes fused quartz, e.g., is pure amorphous silica. Silicon oxide is insoluble in water, slightly soluble in alkalies, and soluble in dilute hydrofluoric acid. Pure silica is colorless to white. Fused silica and fused quartz are distinct from ordinary glass. For example, fused silica or fused quartz does not absorb infrared and ultraviolet light.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

We claim:

1. A syringe comprising a barrel and a plunger;
the barrel defining a cavity, the cavity being configured for containing a fluid;
at least a portion of a fluid contact surface of the cavity comprising a coating of a silicon or siloxane material
the plunger and barrel being configured for drawing fluid into the cavity and expelling liquid from the cavity.

2. The syringe of claim 1, wherein the silicon comprises silicon oxide, silicon carbide, or mixtures thereof.

3. The syringe of claim 1, wherein the siloxane comprises polymethylhydrosiloxane.

4. The syringe of claim 1, wherein the plunger comprises superalloy.

5. The syringe of claim 1, wherein the plunger comprises a fluid contact surface; at least a portion of the fluid contact surface comprising a coating of a silicon or siloxane material.

6. The syringe of claim 5, wherein the silicon comprises silicon oxide, silicon carbide, or mixtures thereof.

7. The syringe of claim 5, wherein the siloxane comprises polymethylhydrosiloxane.

8. The syringe of claim 1, further comprising a needle;
the needle comprising:
superalloy,
a coating of a silicon or siloxane material, or
the coating and the superalloy.

9. A syringe comprising a barrel and a plunger;
the barrel defining a cavity, the cavity being configured for containing a fluid;
the plunger comprising a fluid contact surface;
the plunger comprising superalloy, or
at least a portion of the fluid contact surface comprising a coating of a silicon or siloxane material, or
the plunger comprising the coating and the superalloy;
the plunger and barrel being configured for drawing fluid into the cavity and expelling liquid from the cavity.

10. The syringe of claim 1, wherein the silicon comprises silicon oxide, silicon carbide, or mixtures thereof.

11. The syringe of claim 1, wherein the siloxane comprises polymethylhydrosiloxane.

12. The syringe of claim 1, wherein at least a portion of the surface of the barrel defining the cavity comprises a coating of a silicon or siloxane material.

13. The syringe of claim 12, wherein the silicon comprises silicon oxide, silicon carbide, or mixtures thereof.

14. The syringe of claim 12, wherein the siloxane comprises polymethylhydrosiloxane.

15. The syringe of claim 1, further comprising a needle;
the needle comprising:
superalloy,
a coating of a silicon or siloxane material, or
the coating and the superalloy.

16. An autosampler comprising a syringe; the syringe comprising a barrel and a plunger;
the barrel defining a cavity, the cavity being configured for containing a fluid;
at least a portion of the surface of the barrel defining the cavity comprising a coating of a silicon or siloxane material;
the plunger and barrel being configured for drawing fluid into the cavity and expelling liquid from the cavity;
the autosampler being configured to operate the syringe and to provide a sample to a chromatography apparatus.

17. An autosampler comprising a syringe; the syringe comprising a barrel and a plunger;
the barrel defining a cavity, the cavity being configured for containing a fluid;
the plunger comprising a fluid contact surface;
the plunger comprising superalloy, or
at least a portion of the fluid contact surface comprising a coating of a silicon or siloxane material, or
the plunger comprising the coating and the superalloy;
the plunger and barrel being configured for drawing fluid into the cavity and expelling liquid from the cavity;
the autosampler being configured to operate the syringe and to provide a sample to a chromatography apparatus.

18. A method comprising:
drawing a sample into a syringe, the sample comprising a protic solvent;
the syringe comprising a barrel and a plunger;
the barrel defining a cavity, the cavity being configured for containing a fluid;
at least a portion of the surface of the barrel defining the cavity comprising a coating of a silicon or siloxane material;
the plunger and barrel being configured for drawing fluid into the cavity and expelling liquid from the cavity.

19. The method of claim 18, further comprising introducing the sample into a chromatography system.

20. A method comprising:
drawing a sample into a syringe, the sample comprising a protic solvent;
the syringe comprising a barrel and a plunger;
the barrel defining a cavity, the cavity being configured for containing a fluid;
the plunger comprising a fluid contact surface;
the plunger comprising superalloy, at least a portion of the fluid contact surface comprising a coating of a silicon or siloxane material, or the plunger comprising the coating and the superalloy.

21. The method of claim 20, further comprising introducing the sample into a chromatography system.
